(19) United States
(12) Patent Application Publication
(10) Pub. No.: US 2003/0019781 A1
(43) Pub. Date: Jan. 30, 2003

(54) Capsule container system (CCS)

(76) Inventor: Robert William Kocher, Arlington, VA (US)

Correspondence Address: ROBERT W. KOCHER, 4828 3rd St. NORTH, ARLINGTON, VA, 22203, US

(21) Appl. No.: 09/916,748

(22) Filed: Jul. 30, 2001

(51) Int. Cl. A61K 9/48; B65D 83/04; B65D 85/42

(52) U.S. Cl. 206/539

(57) ABSTRACT

The capsule container system (CCS) invention relates to a small, single-use, disposable container having various configurations, which can durably be carried in a pocket and containing solutions or substance with easy opening capability to allow the user to dispense the substance. This invention replaces current small plastic bottles that are currently carried in pockets or purses containing various substances.

### CROSS REFERENCE TO RELATED APPLICATIONS

Not Applicable

### STATEMENT REGUARDING FEDERALLY SPONSORED RESEARCH OR DEVELOPMENT

Not Applicable

### REFERENCE TO A MICROFICHE APPENDIX

Not Applicable

### BACKGROUND-PRIOR ART

Small plastic bottles or tubes are the most common container devices for carrying liquid or cream substances in a pocket. These devices typically require an enclosure or top to seal the container. Many devices are too large to inconspicuously fit in a pocket. Devices, such as bottles or tubes with a screw off cap contribute a significant proportion to the cost of the product.

Substances such as hand sanitation solutions, hand creams, tooth past, suntan lotion, and lubricants are typically found in relative large bottles and tubes which make them difficult to carry in a pocket or just have one serving.

Bottles are cumbersome to carry in a pocket and are intended for anywhere from 25 to 200 uses. These containers require a relatively large cap for opening and closing. Once opened there is the risk of a substantial quantity of leakage due to their high volume compared to a single use requirement. These bottles also require two hands to operate. Multi-use containers also require the passing from one person to another, again risking contamination on the outside of the bottle. Multi-use containers pose the challenge of dispensing the right quantity. Normally one must be very careful not to dispense too much. You must quickly focus on what you are doing and attempt to get the right amount out of the bottle.

U.S. Pat. No. 5,063,057 by Spellman, et all, entitled Cosmetic Capsules addresses the small packaging issues and requirements for cosmetic products. The capsule container system (CCS) specific packaging configurations significantly differ in structure from the structure shown in U.S. Pat. No. 5,063,057.

U.S. Pat. No. 6,228,375 by Kocher, entitled Micro Hand Sanitizers address a version of the capsule for of this invention. This invention expands the use of the container to other liquids and creams outside hand sanitation.

### OBJECTIVES AND ADVANTAGES

The objective of the capsule container system (CCS) is to provided and more efficient method of carrying and using solutions, creams, lotions in public environments. The capsule container system (CCS) has significant advantages over current methods.

(a) The CCS is significantly smaller than containers with a substance volume of approximately 0.5 to 5.0 milliliters, just enough for a single use. This small size allows the CCS to be carried in a shirt, pants pocket, coat pocket, or larger quantities in a briefcase, or purse.

(b) The CCS's individual packaging allows the user to carry the quantity deemed necessary in a pocket and the remainder in the desk, briefcase or purse. The individual packaging also allows for a bowl or jar of CCS to be placed in areas where multiple people have needs.

(c) The CCS is a single use system therefore the simple squeezing of a capsule squirts the right amount. CCS's single use size does not require the slow, careful watching of the quantity being dispensed. The user can focus on something else, such as watching a computer screen, watching the road while driving, involved in another activity.

(d) The CCS can be made of a biodegradable material that is environmentally friendly.

### DRAWING FIGURES

FIG. 1. shows a CCS capsule configuration with a notched tip serving as a closure for the encapsulating container.

FIG. 2. shows a partial view of a single notch in the tip.

FIG. 3. shows the capsule having applied pressure (arrows) and the ruptured notched tip.

FIG. 4. is a capsule configuration with a capped dispensing hole serving as a closure.

FIG. 5. shows a capsule configuration with a tape seal over a slit dispensing hole.

FIG. 6. shows a capsule configuration with two interlocking halves.

FIG. 7. shows a sphere configuration with a tape cover over a dispensing hole.

FIG. 8. shows a disk configuration with a tape cover over a dispensing hole.

FIG. 9. shows a disk configuration with a tape cover over a dispensing hole on the edge.

FIG. 10. shows a tube configuration with a tap cover over a dispensing slit.

FIG. 11. shows a tube configuration with pinched ends.

FIG. 12. shows a tube configuration with ends bent over.

### REFERENCE NUMERALS IN DRAWINGS

1. Capsule Configuration

2. Notch or Cut

3. Dispensing Hole Plug

4. Tape Covering Dispensing Hole

5. Tab to Pull or Lift-off Tape

6. Pre-cut Dispensing Hole or Slit

7. Outer Half of Two Piece Capsule

8. Inner Half of Two Piece Capsule

9. Sphere/Ball/“Egg” Configuration

10. Disk or Coin Configuration

11. Tube Configuration

12. Crimped End of Tube

13. Folded End of Tube

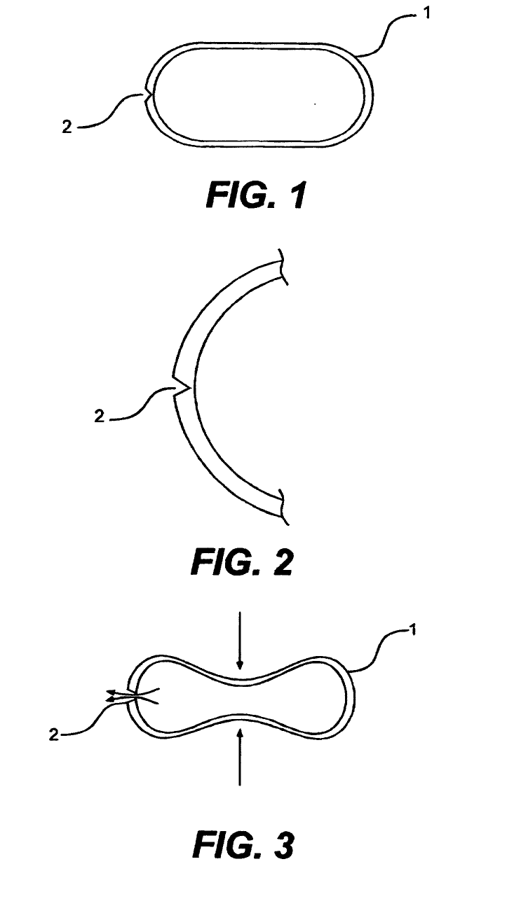

### DESCRIPTION—FIGS. 1 to 3—PREFERRED EMBODIMENT

A preferred embodiment of the capsule container system (CCS) is a soft crushable material capsule with a volumetric capacity of approximately 0.25 to 5.0 milmiters of solution, creams or pasts. For example, a commonly used solution is hand cream, suntan lotions, or petroleum jelly are contemplated for using in my invention. Other custom formulas are envisioned. FIG. 1 depicts the general elongated capsule configuration. The capsule can be fitted with several different dispensing approaches. The first of these methods is shown in FIGS. 1-3. Item 2 depicts a small notch or semi-perforated hole made in the tip of the capsule during the manufacturing process. This hole or notch is cut deep enough such that when the sides of the capsule are squeezed hard between two fingers, the pre-cut area will rupture spraying the contents in the intended direction. The pre-cut end can be marked with an arrow or colored tip to make it easy for the user to identify out of which end the contents will spray.

FIG. 2 depicts a close up view of Item 2, the notch.

FIG. 3 shows pressure being applied to the sides of the capsule and the pre-cut surface rupturing, spraying the contents out in one squirt of the collapsing capsule.

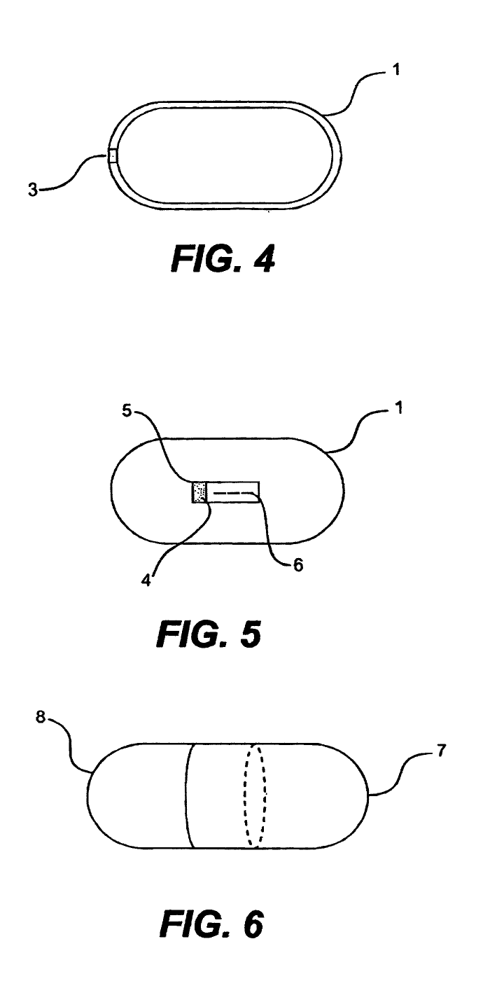

FIGS. 4-6 Alternative Embodiments (Capsule)

FIG. 4 depicts a capsule configuration with pre-cut hole and cap configuration in the end of the capsule. Item 3 is a cap that is inserted in the pre-cut hole during the manufacturing process. This cap can be released either through pressure or can be scratched off with a fingernail. This dispensing configuration would allow single hand operation.

FIG. 5 depicts a capsule configuration with a pre-cut slit or hole, Item 6, covered with a tape, Item 4. During the manufacturing process, a small slit or hole is made in the capsule then covered with a tape or plastic cover. The tape, Item 4, can have a tab on the end to allow ease in prying up the end of the tape. A plastic cover could be pried up with a fingernail to allow one hand opening and dispensing of the solution.

FIG. 6 depicts a capsule configuration consisting of two halves, Item 7 and Item 8, whereby one half has a slightly smaller diameter and fits inside the other half. The dispensing mechanism is simply the separation of the two halves and pouring the solution into the hands.

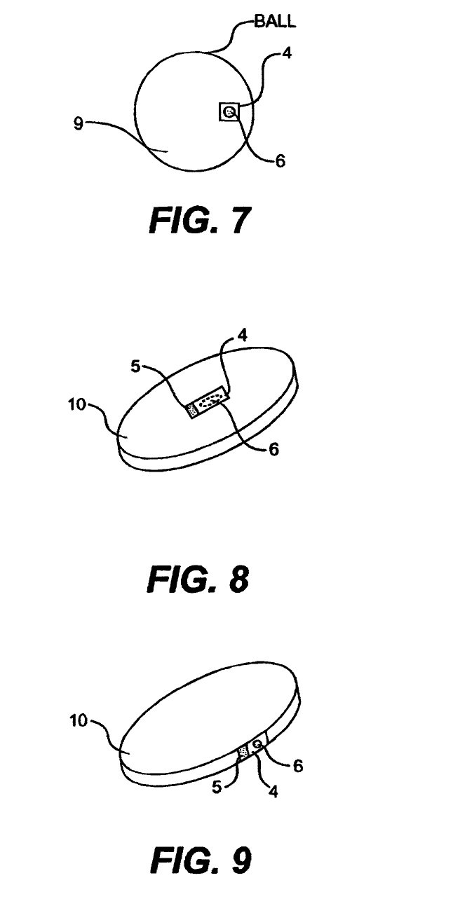

FIG. 7 Alternative Embodiments (Sphere)

FIG. 7 depicts a sphere, ball, or oblong egg configuration whereby a precut hole is made in the surface, Item 6, and covered with a tape or plastic. The tape or plastic can have a tab to allow ease in prying the cover off. Once the cover is off, the sphere is crushed and the solution is sprayed through the pre-cut hole. The notched or semi-perforated hole method could also be used.

FIGS. 8-9 Alternative Embodiments (Disk)

FIG. 8 depicts a disk shaped enclosure, Item 10, with a pre-cut hole, Item 6, covered by a tape or plastic cover, Item 4, with an optional tab, Item 5. This disk configuration can resemble a coin to be carried along with pocket change.

FIG. 9 depicts a disk shaped enclosure with the closure and dispensing hole on the edge of the disk.

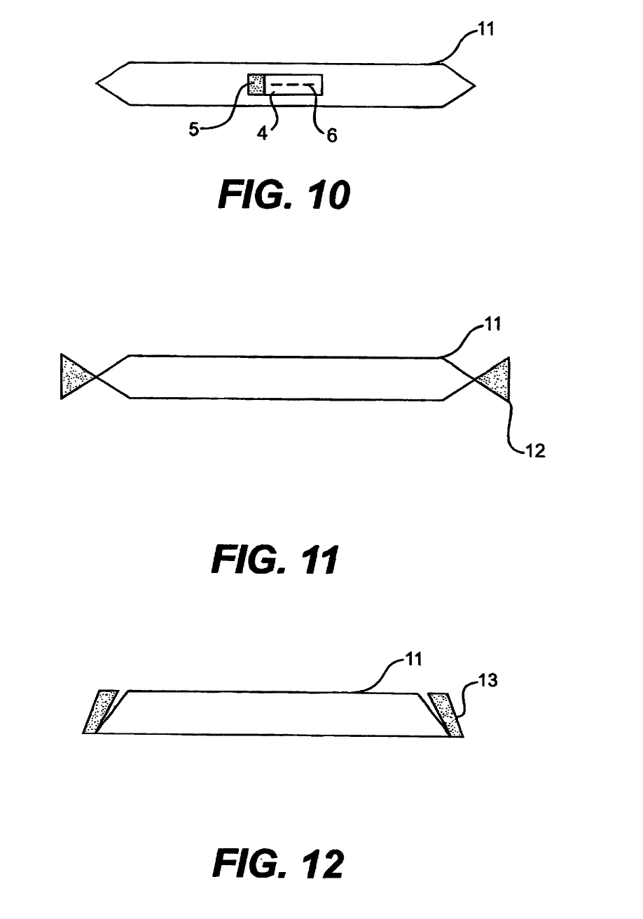

FIGS. 10-12 Alternative Embodiments (Tubes)

FIG. 10 depicts a tube shaped enclosure, Item 11. A tube shaped enclosure constructed from a small tube capable of holding less than 2.0 milliliters. FIG. 11 depicts the tube with sealed ends, a pre-cut slot, Item 6 covered by a tape or plastic, Item 4, with a tab, Item 6.

FIG. 11 depicts a tube shaped with sealed ends, Item 12, that are clamped closed to seal in the solution. Once the clamp is removed the end becomes the dispensing opening.

FIG. 12 depicts a tube shaped enclosure, Item 11 with the ends folded back on itself Item 13, to seal in the solution. An adhesive can be applied to hold down the folded end. The folded end is opened by breaking the glue seal and folding back the end and squeezing the tube to dispense the solution though the end of the tube.

Advantages

From the description above, a number of advantages of the capsule container system (CCS) can be realized. The CCS allows for the practicable carrying of solutions in single use quantities of about 0.25 to 5.0 milliliters in pockets where conventional bottles do not fit. Several of the CCS's dispensing system allows dispensing in a fraction of the time it takes conventional bottle, hand washing or towel systems. Several of the CCS's single hand opening techniques allow for inconspicuous use in crowded area.

Operation—FIGS. 1-3 Capsule with Semi-Perforated Tip

Hold the capsule in one hand, point at the other hand and squeeze the capsule until the semi-perforated tip bursts and sprays the solution onto the other hand. Toss the capsule away.

Operation—FIG. 4 Capsule with Plug Tip

One hand operation: This can be done in your pocket. Hold the capsule in one hand and with you finger nail pry off the tip plug. Squeeze the capsule dispensing the solution in the same hand. Toss away the capsule. The solution in now in one hand.

Operation—FIG. 5, 7-10, Capsule, Sphere/Ball, Disk, and Tube.

One hand operation: This can be done in your pocket or under a table. Hold the enclosure in one hand with the cover tab towards you thumb. Pry off the cover with your fingernail. Squeeze the enclosure until the substance dispenses into your hand.

Two-hand operation: This is done when you are able to use two hands or when the cover is a tape with tab. Hold the enclosure in one hand. With the other hand, grab the tab and pull off the cover.

Operation—FIG. 6 Capsule with Two Halves

With both hands, pull the capsule apart. Squeeze the halves into the hands.

Operation—FIG. 11 Tube with Clamped Ends

With both hands, remove the clamp and squeeze the tube dispensing the solution into one hand.

Operation—FIG. 12 Tube with Folded Ends

One hand operation: With one hand, hold the tube and with the thumbnail pry open the folded end then turn the tube around and squeeze solution into the hand.

### CONCLUSION, RAMIFICATIONS, AND SCOPE.

The reader can see that the capsule container system CCS substantially differs from current approaches because it is single use with:

volumetric capacity less that 5 milliliters,

simple, rapid dispensing methods.

These differences from conventional methods allow for the CCS to be carried in shirt pockets, pants pockets or confined areas where bottles won't fit. It allows the user to carry one “just in case”.

The single use feature:

Allows for the simple designs because resealing is not required;

also avoids the requirement to watch and determine how much is being dispensed; and,

allows for bulk quantities to be placed in areas of public use.

The CCS can be used in an inconspicuous way in a crowd of people. The one hand opening method discussed allow for opening in a pocket or under a desk that does not require one's full attention. The user can still focus on ongoing activities.

The CCS can be used in a limited sense to place substances on doorknobs, telephone handsets, pens, or other high use small items.

The ramifications of the capsule container system will be significant in today society where a significant number of people carry liquids, jells, creams, for individual and single use.

Thus the scope of the capsule container system invention should be determined by the appended claims and their legal equivalents, rather than by the examples given.

What I claim as my invention is:

1. A capsule container system comprising:
an encapsulating container sized to be held and opened between two fingers and composed of a disposable material which is capable of only single use;
a substance contained within the miniature encapsulating container;
said encapsulating container holding about 0.25 to 5.0 milliliters of said substance;
said encapsulating container has a shape selected from the group consisting of a sphere, cylinder, capsule and disk; and,
an encapsulated opening system located on one end or location on said encapsulating container selected from the group consisting of configurations using a semi-perforated hole, incision or a manufactured thin area, which allows quick and directional dispensing of said substance once the container has been opened.

2. The container system of claim 1, where the container is comprised of a material selected such that sufficient squeezing force causes the container to burst open at a predetermined location.

3. The container system of claim 1, where the container is comprised of an environmental compatible materiel that will decompose after use.

4. A method of dispensing a substance into a person's hands and other small areas, comprising the steps of obtaining a container containing a substance and sized to be concealable with one hand of the person, holding the container by two fingers of the one hand or within the palm of the one hand, opening the container with the one hand or with at least one finger of the person's other hand, and dispensing substantially all of the substance in the container to the person's one hand or the other hand or to a small area on another object.
